(19) United States
(12) Patent Application Publication
(10) Pub. No.: US 2020/0024650 A1
(43) Pub. Date: Jan. 23, 2020

(54) Target Polynucleotide Detection and Sequencing by Incorporation of Modified Nucleotides for Nanaopore Analysis

(71) Applicant: Ontera Inc., Santa Cruz, CA (US)

(72) Inventors: Stephen P. Hendricks, Los Gatos, CA (US); Trevor J. Morin, Santa Cruz, CA (US)

(21) Appl. No.: 16/585,579

(22) Filed: Sep. 27, 2019

Related U.S. Application Data
(63) Continuation of application No. PCT/US2018/024636, filed on Mar. 27, 2018.
(60) Provisional application No. 62/477,965, filed on Mar. 28, 2017.

(51) Int. Cl. C12Q 1/6825; C12Q 1/6876; G01N 33/487

(52) U.S. Cl. CPC C12Q 1/6825; C12Q 2600/156; G01N 33/48721; C12Q 1/6876

(57) ABSTRACT

Disclosed herein are methods and compositions for target polynucleotide identification by using modified nucleotides incorporated into the polynucleotide to facilitate detection via a nanopore device.

### CROSS-REFERENCE TO RELATED APPLICATIONS

This application is a continuation of international application PCT/US2018/024636 filed Mar. 27, 2018, which claims the benefit of U.S. Provisional Application No. 62/477,965, filed Mar. 28, 2017, the entire disclosure of each is hereby incorporated by reference in their entirety for all purposes.

### FIELD OF THE INVENTION

The invention relates to methods and compositions for target polynucleotide identification using modified nucleotides to facilitate detection via a nanopore device.

### BACKGROUND OF THE INVENTION

In some applications in which DNA is amplified prior to nanopore detection, labeling of the DNA may be useful to increase detectability, sensitivity and/or specificity of the nanopore signal. This labeling can be done by hybridizing sequence-specific probes to the newly synthesized DNA. However, this approach may not be ideal for all applications since hybridization probes may be difficult to design for some target sequences and require additional steps and special conditions for optimal hybridization of probe to target DNA. In addition, hybridization does not result in covalent attachment of probe to target DNA. Therefore, one must maintain conditions during the nanopore measurements that favor stable hybridization of probe to target DNA. For these and other reasons, alternate methods for labeling target DNA or other polynucleotides are desired in nanopore detection applications.

### SUMMARY OF THE INVENTION

The instant invention is based, at least in part, on the discovery that incorporation of modified nucleotides during nucleic acid polymerization results in the addition of nanopore detection labels that enhance the detectability of the newly synthesized nucleic acid in a nanopore device. This form of labeling can provide certain advantages over conventional methods, including the ability to detect shorter amplicons during nanopore detection, an improved resolution of multiplexed detection from a sample, and higher resolution of detection as compared to previous nanopore detection methods.

Accordingly, in some embodiments, provided herein are methods to detect a target polynucleotide by performing a sequence-specific reaction to incorporate one or more modified nucleotides into a polynucleotide, and detecting the reaction products via a nanopore device.

Also provided herein, according to some embodiments, is a method of determining the presence or absence of a target polynucleotide suspected of being present in a sample, comprising: providing a sample suspected of comprising a target polynucleotide; providing one or more modified nucleotides; performing a template-driven polymerization reaction on said sample to incorporate said one or more modified nucleotides into a growing strand complementary to said target polynucleotide, if present, to generate an polynucleotide reaction product comprising said one or more modified nucleotides; loading said sample into a device comprising a nanopore, wherein said nanopore separates an interior space of the device into two volumes; configuring the device to pass the polynucleotide reaction product, if present, through said nanopore, wherein the device comprises a sensor configured to detect an electrical signal generated by objects passing through the nanopore; and detecting the presence or absence of said target polynucleotide in said sample by determining whether a polynucleotide comprising said one or more modified nucleotides passed through the nanopore using said electrical signal from the sensor.

In some embodiments, the one or more modified nucleotides comprises a direct label modified nucleotide or an indirect label modified nucleotide. In some embodiments, the one or more modified nucleotides comprises a charged chemical moiety, a neutral chemical moiety, a hydrophobic moiety, or a hydrophilic moiety. In some embodiments, the one or more modified nucleotides comprises a linker capable of binding to a charged chemical moiety, a neutral chemical moiety, a hydrophobic moiety, or a hydrophilic moiety.

In some embodiments, the one or more modified nucleotides comprises a fluorescent dye. In some embodiments, the one or more modified nucleotides comprises Atto488, Atto425, Atto532, Cy5, Texas Red, Fluorescein-12, Rhodamine-12, or aminomethylcoumarin-6. In some embodiments, the modified nucleotide is a dUTP, dTTP, dCTP, dATP, or dGTP.

In some embodiments, the one or more modified nucleotides comprises a polyethylene glycol. In some embodiments, the one or more modified nucleotides comprise a linker. In some embodiments, the linker comprises bromo-2′-deoxyuridine-5′-triphosphate (BrdUTP), 5-aminoallyl-2′-deoxyuridine-5′-triphosphate, or 5-ethynyl-2′-deoxyuridine-5′-triphosphate (EdUTP), N6-(6-amino)hexyl-dATP (or dUTP or dCTP) and 7-propargylamino-7-deaza-dATP (or dUTP or dCTP).

In some embodiments, the method of determining the presence or absence of a target polynucleotide suspected of being present in a sample further comprises binding a detectable moiety to said linker, wherein said electrical signal is modified by the presence of said detectable moiety. In some embodiments, the detectable moiety comprises an antibody. In some embodiments, the detectable moiety comprises N-hydroxysuccinimide. In some embodiments, the detectable moiety is azide-modified.

In some embodiments, the sensor measures an electrical signal that fluctuates upon translocation of said polynucleotide reaction product through said nanopore.

In some embodiments, the method of determining the presence or absence of a target polynucleotide suspected of being present in a sample comprises detecting a plurality of target polynucleotides suspected of being present in the same sample by generating a plurality of distinct polynucleotide reaction products. In some embodiments, the electrical signal is distinct for each of said plurality of target polynucleotides. In some embodiments, the plurality of distinct polynucleotide reaction products are of different lengths. In some embodiments, the plurality of distinct polynucleotide reaction products each comprise a unique modified nucleotide. In some embodiments, the plurality of distinct polynucleotide reaction products comprise a unique modified nucleotide incorporation pattern. In some embodiments, the template-driven polymerization reaction comprises a plurality of sequence-specific primers to enable multiplexed amplification and nanopore detection of more than one target.

In some embodiments, the polynucleotide is DNA or RNA. In some embodiments, the template-driven polymerization reaction is an amplification reaction. In some embodiments, the amplification reaction is a polymerase chain reaction or an isothermal reaction. In some embodiments, the template-driven polymerization reaction comprises a polymerase. In some embodiments, the polymerase is DNA polymerase or a reverse transcriptase. In some embodiments, the polymerase is a thermostable DNA polymerase or a thermolabile DNA polymerase.

In some embodiments, the modified nucleotide comprises a plurality of labels. In some embodiments, the polynucleotide reaction product is from 200 to 500 bases, from 100 to 2,000 bases, or from 50 to 10,000 bases in length. In some embodiments, the polynucleotide reaction product is greater than 50 bases, greater than 100 bases, greater than 200 bases, greater than 300 bases, or greater than 400 bases in length. In some embodiments, the polynucleotide reaction product is less than 50,000 bases, less than 10,000 bases, less than 5,000 bases, less than 1,000 bases, or less than 500 bases in length.

Also provided herein, according to some embodiments, is a method of identifying a modified nucleotide present in a polynucleotide, comprising: loading a sample suspected of comprising a polynucleotide comprising a plurality of modified nucleotides into a device comprising a nanopore, wherein said nanopore separates an interior space of the device into two volumes; configuring the device to pass the polynucleotide, if present, through said nanopore, wherein the device comprises a sensor configured to detect an electrical signal generated by objects passing through the nanopore, and wherein said polynucleotide comprising said modified nucleotides generates a distinct electrical signal from a polynucleotide without said modified nucleotides; and detecting the presence or absence of said polynucleotide in said sample by determining whether said polynucleotide comprising said plurality of modified nucleotides passed through the nanopore using said electrical signal detected by the sensor.

In some embodiments, the polynucleotide comprises at least 5, at least 10, at least 20, at least 30, at least 40, at least 50, at least 100, at least 200, or at least 500 modified nucleotides. In some embodiments, the polynucleotide is DNA or RNA.

In some embodiments, the plurality of modified nucleotides comprise a direct label modified nucleotide or an indirect label modified nucleotide. In some embodiments, the plurality of modified nucleotides comprise a charged chemical moiety, a neutral chemical moiety, a hydrophobic moiety, or a hydrophilic moiety. In some embodiments, the plurality of modified nucleotides comprise a linker capable of binding to a charged chemical moiety, a neutral chemical moiety, a hydrophobic moiety, or a hydrophilic moiety. In some embodiments, the plurality of modified nucleotides comprise a fluorescent dye. In some embodiments, the plurality of modified nucleotides comprise Atto488, Atto425, Atto532, Cy5, Texas Red, Fluorescein-12, Rhodamine-12, or aminomethylcoumarin-6. In some embodiments, the plurality of modified nucleotides comprise dUTP, dTTP, dCTP, dATP, or dGTP. In some embodiments, the plurality of modified nucleotides comprise a polyethylene glycol. In some embodiments, the plurality of modified nucleotides comprise a linker. In some embodiments, the linker comprises bromo-2′-deoxyuridine-5′-triphosphate (BrdUTP), 5-aminoallyl-2′-deoxyuridine-5′-triphosphate, or 5-ethynyl-2′-deoxyuridine-5′-triphosphate (EdUTP), N6-(6-amino)hexyl-dATP (or dUTP or dCTP) and 7-propargylamino-7-deaza-dATP (or dUTP or dCTP).

In some embodiments, the method of identifying a modified nucleotide present in a polynucleotide further comprises binding a detectable moiety to said linker, wherein said electrical signal is modified by the presence of said detectable moiety. In some embodiments, the detectable moiety comprises an antibody. In some embodiments, the detectable moiety comprises N-hydroxysuccinimide. In some embodiments, the detectable moiety is azide-modified.

In some embodiments, the sensor measures an electrical signal that fluctuates upon translocation of said polynucleotide reaction product through said nanopore.

In some embodiments, the method of identifying a modified nucleotide present in a polynucleotide comprises detecting a plurality of distinct polynucleotides comprising modified nucleotides. In some embodiments, the electrical signal is distinct for each of said plurality of polynucleotides. In some embodiments, the plurality of distinct polynucleotides are of different lengths. In some embodiments, the plurality of distinct polynucleotides each comprise a unique modified nucleotide. In some embodiments, the plurality of distinct polynucleotides have a distinct pattern of modified nucleotides along the length of each distinct polynucleotide.

In some embodiments, the modified nucleotide comprises a plurality of labels.

In some embodiments, the polynucleotide is from 200 to 500 bases, from 100 to 2,000 bases, or from 50 to 10,000 bases in length. In some embodiments, the polynucleotide is greater than 50 bases, greater than 100 bases, greater than 200 bases, greater than 300 bases, or greater than 400 bases in length. In some embodiments, the polynucleotide is less than 50,000 bases, less than 10,000 bases, less than 5,000 bases, less than 1,000 bases, or less than 500 bases in length.

In some embodiments, the modified nucleotides are individually incorporated into said polynucleotide by an amplification reaction.

Also provided herein is a system comprising a device comprising a nanopore, wherein said nanopore separates an interior space of the device into two volumes, wherein the device comprises a sensor configured to detect an electrical signal generated by objects passing through the nanopore; a polynucleotide comprising a modified nucleotide, wherein said polynucleotide is loaded into said device for detection by voltage-induced translocation through said nanopore; and a module for analyzing said electrical signal to detect the presence or absence of said polynucleotide comprising said modified nucleotide.

In some embodiments, the polynucleotide comprises a plurality of modified nucleotides. In some embodiments, the polynucleotide is DNA or RNA. In some embodiments, the polynucleotide is from 200 to 500 bases, from 100 to 2,000 bases, or from 50 to 10,000 bases in length.

In some embodiments, the polynucleotide comprises at least 5, at least 10, at least 20, at least 30, at least 40, at least 50, at least 100, at least 200, or at least 500 modified nucleotides.

In some embodiments, the modified nucleotide comprises a direct label modified nucleotide or an indirect label modified nucleotide. In some embodiments, the modified nucleotide comprises a charged chemical moiety, a neutral chemical moiety, a hydrophobic moiety, or a hydrophilic moiety. In some embodiments, the modified nucleotide comprises a linker capable of binding to a charged chemical moiety, a neutral chemical moiety, a hydrophobic moiety, or a hydrophilic moiety. In some embodiments, the modified nucleotide comprises a fluorescent dye. In some embodiments, the modified nucleotide comprises Atto488, Atto425, Atto532, Cy5, Texas Red, Fluorescein-12, Rhodamine-12, or aminomethylcoumarin-6. In some embodiments, the plurality of modified nucleotide is dUTP, dTTP, dCTP, dATP, or dGTP. In some embodiments, the modified nucleotide comprises a polyethylene glycol.

In some embodiments, the modified nucleotide comprise a linker. In some embodiments, the linker comprises bromo-2′-deoxyuridine-5′-triphosphate (BrdUTP), 5-aminoallyl-2′-deoxyuridine-5′-triphosphate, or 5-ethynyl-2′-deoxyuridine-5′-triphosphate (EdUTP), N6-(6-amino)hexyl-dATP (or dUTP or dCTP) and 7-propargylamino-7-deaza-dATP (or dUTP or dCTP). In some embodiments, the linker is bound to a detectable moiety, and said electrical signal is modified by the presence of said detectable moiety. In some embodiments, the detectable moiety comprises an antibody. In some embodiments, the detectable moiety comprises N-hydroxysuccinimide. In some embodiments, the detectable moiety is azide-modified.

In some embodiments, the sensor is configured to measure an electrical signal that fluctuates upon translocation of said polynucleotide reaction product through said nanopore.

In some embodiments, the modified nucleotide comprises a plurality of labels.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1A illustrates the structure of Atto488 dUTP, which can be used as a direct label modified nucleotide to facilitate detection of a target polynucleotide in a nanopore, according to an embodiment of the invention.

FIGS. 1A-1C depict embodiments of modified nucleotides comprising labels for detection in a nanopore. Specifically shown are the dUTP-label constructs for incorporation into DNA as substitutes for dTTP. FIG. 1A shows dUTP-Atto-488. FIG. 1B shows dUTP-Cy5. FIG. 1C shows dUTP-Atto-532.

FIG. 2 illustrates a reaction scheme of incorporation of modified nucleotides into a target polynucleotide, followed by binding of labels to said modified nucleotides for subsequent detection in a nanopore, according to an embodiment of the invention. In panel A), a sample containing DNA is mixed with amplification reaction components including ethynyl dUTP and incubated under conditions that promote DNA amplification. In panel B), following the amplification reaction, the newly synthesized DNA will contain incorporated EdU. In panel C), the newly synthesized DNA is exposed to an azide-modified detection reagent, which undergoes the “CLICK” reaction resulting in labeled DNA.

FIG. 3 provides a DNA amplification product labeled with linear polyethylene glycol (PEG), according to an embodiment of the invention.

FIGS. 4A and 4B show comparisons of nanopore event populations for 279bp alone and 279bp-dUTP-Atto-488. FIG. 4A shows the event plot of each population. FIG. 4B shows a comparison of the percentage of events having a max dG greater than 1 nS that is used to detect the presence of the target polynucleotide including the dUTP-Atto-488 modified nucleotide with 99% confidence.

FIGS. 5A and 5B show comparisons of nanopore event populations for 466bp alone and 466bp-dUTP-Atto-488. FIG. 5A shows the event plot of each population. FIG. 5B shows a comparison of the percentage of events having a max dG greater than 1 nS that is used to detect the presence of the target polynucleotide including the dUTP-Atto-488 modified nucleotide with 99% confidence.

FIG. 6 shows the results of an electrophoretic mobility shift assay (EMSA) of amplification products used in nanopore experimentation. FIG. 6 depicts 279bp DNA without modification (lane 2) in addition to 279bp with dUTP-Atto-488 or Atto-532 incorporated (lanes 3 and 4 respectively). A shift up in the gel matrix indicates successful incorporation of dUTP modifications. Successful incorporation of dUTP-Atto-488 in a 466bp amplicon is also shown (lane 6).

FIGS. 7A and 7B show comparisons of nanopore event populations for 279bp alone and 279bp-dUTP-Cy5. FIG. 7A shows the event plot of each population. FIG. 7B shows a comparison of the percentage of events having a max dG greater than 1 nS that is used to detect the presence of the target polynucleotide including the dUTP-Cy5 modified nucleotide with 99% confidence.

FIGS. 8A and 8B show comparisons of nanopore event populations for 466bp alone and 466bp-dUTP-Atto-532. FIG. 8A shows the event plot of each population. FIG. 8B shows a comparison of the percentage of events having a max dG greater than 1 nS that is used to detect the presence of the target polynucleotide including the dUTP-Atto-532 modified nucleotide with 99%

FIG. 9 shows the results of an electrophoretic mobility shift assay (EMSA) of amplification products used in nanopore experimentation. FIG. 9 shows 279bp DNA without modification (lane 2) and the same 279bp DNA with dUTP-Cy5 incorporated (lane 3).

### DETAILED DESCRIPTION

According to some embodiments of the invention, provided herein is a method to incorporate modified nucleotides during nucleic acid polymerization or amplification to synthesize target DNA molecules comprising one or more detection labels suitable for detection upon translocation through or capture by a nanopore. In some embodiments, these labels will be distributed in a regular, symmetric fashion throughout the newly synthesized DNA. Labeling in this manner can enhance the detectability of these molecules in the nanopore. This approach can also increase the resolution of detection of short DNA amplicons of lengths that are not typically detectable in a nanopore device without labels. In some embodiments, multiplexed target detection can be accomplished by creating unique length amplicons for each target of interest.

The details of various embodiments of the invention are set forth in the description below. Other features, objects, and advantages of the invention will be apparent from the description and the drawings, and from the claims.

Throughout this application, the text refers to various embodiments of the present devices, compositions, systems, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entireties

As used herein, the term “comprising” is intended to mean that the systems, devices, and methods include the recited components or steps, but not excluding others. “Consisting essentially of when used to define systems, devices, and methods, shall mean excluding other components or steps of any essential significance to the combination. “Consisting of shall mean excluding other components or steps. Embodiments defined by each of these transition terms are within the scope of this invention.

All numerical designations, e.g., distance, size, temperature, time, voltage and concentration, including ranges, are approximations which are varied (+) or (−) by increments of 0.1. It is to be understood, although not always explicitly stated that all numerical designations are preceded by the term “about”. It also is to be understood, although not always explicitly stated, that the components described herein are merely exemplary, and that equivalents of such are known in the art.

As used herein, “a device comprising a nanopore that separates an interior space” shall refer to a device having a pore that comprises an opening within a structure, the structure separating an interior space into two volumes or chambers. The device can also have more than one nanopore, and with one common chamber between every pair of pores.

The term “polynucleotide” as used herein refers to a polymeric form of nucleotides of any length, either ribonucleotides or deoxyribonucleotides. This term refers only to the primary structure of the molecule. Thus, this term includes double- and single-stranded DNA and RNA.

As used herein, the term “target polynucleotide” refers to a polynucleotide comprising a sequence of interest (i.e., a target polynucleotide sequence or a target sequence). A target polynucleotide can include regions (e.g., sufficiently complementary sequences) for hybridizing to primers for amplification of the target polynucleotide. These regions can be part of the sequence of interest, flanking the sequence of interest, or further upstream or downstream of the sequence of interest in sufficient proximity to allow amplification of the sequence of interest via an amplification reaction. In some embodiments, these regions for hybridizing to primers are located at the two ends of the amplicon generated by an amplification reaction. Described herein according to some embodiments are methods, devices, and compositions for detecting a target polynucleotide comprising a sequence of interest.

As used herein, the term “amplification” or “amplification reaction” refers to a reaction that generates a plurality of clonal amplicons comprising a target polynucleotide sequence from the target polynucleotide sequence. As used herein, amplification reaction reagents include any molecules that are necessary to perform amplification of the target polynucleotide sequence. Amplification reaction reagents can include, but are not limited to, free primers, dNTPs (deoxynucleotide triphosphates, dATP, dGTP, dCTP, dTTP), polymerase enzymes (e.g., Taq or Pfu), salts (Magnesium chloride, Magnesium Sulfate, Ammonium sulfate, sodium chloride, potassium chloride), BSA (bovine serum albumin) stabilizer, and detergents (e.g., triton X-100). Amplification reactions can include, but are not limited to, e.g., PCR, ligase chain reaction (LCR), transcription mediated amplification (TMA), reverse transcriptase initiated PCR, DNA or RNA hybridization techniques, sequencing, isothermal amplification, and loop-mediated isothermal amplification (LAMP). Techniques of amplification to generate an amplicon from a target polynucleotide sequence are well known to one of skill in the art.

As used herein, the term “nanopore” refers to an opening (hole or channel) of sufficient size to allow the passage of particularly sized polymers. With an amplifier, voltage is applied to drive negatively charged polymers through the nanopore, and the current through the pore detects if molecules are passing through it.

As used herein, the term “sensor” refers to a device that collects a signal from a nanopore device. In many embodiments, the sensor includes a pair of electrodes placed at two sides of a pore to measure an ionic current across the pore when a molecule or other entity, in particular a polymer scaffold, moves through the pore. In addition to the electrodes, an additional sensor, e.g., an optical sensor, may be to detect an optical signal in the nanopore device. Other sensors may be used to detect such properties as current blockade, electron tunneling current, charge-induced field effect, nanopore transit time, optical signal, light scattering, and plasmon resonance.

As used herein, the term “current measurement” refers to a series of measurements of current flow at an applied voltage through the nanopore over time. The current is expressed as a measurement to quantitate events, and the current normalized by voltage (conductance) is also used to quantitate events.

As used herein, the term “open channel” refers to the baseline level of current through a nanopore channel within a noise range where the current does not deviate from a threshold of value defined by the analysis software.

As used herein, the term “event” refers to a set of current impedance measurements that begins when the current measurement deviates from the open channel value by a defined threshold, and ends when the current returns to within a threshold of the open channel value.

### Modified Nucleotides

Many types of modified nucleoside triphosphates are available commercially that are readily incorporated into the newly synthesized strand of DNA during template-dependent nucleic acid amplification. The basis of this application is that incorporation of these modified nucleotides during nucleic acid amplification results in the addition of nanopore detection labels that enhance the detectability of the newly synthesized nucleic acid. For the purposes of this application the modified nucleotides can be divided into two groups, direct label nucleotides and indirect label nucleotides.

Direct Label Modified Nucleotides

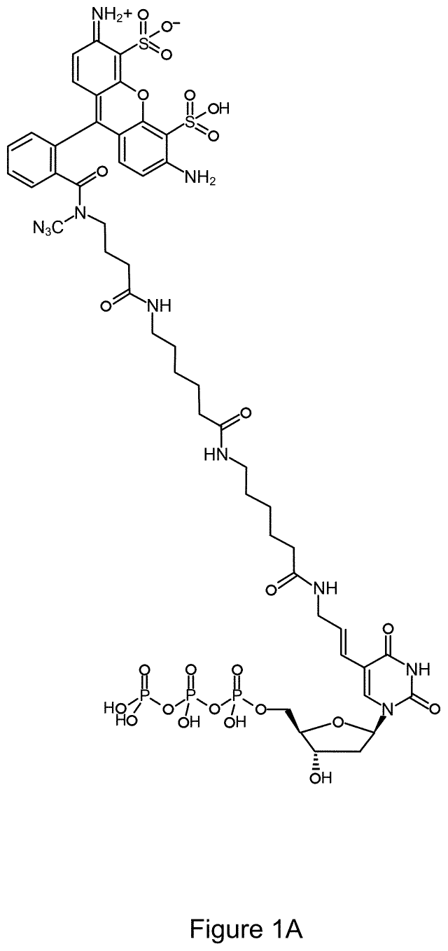

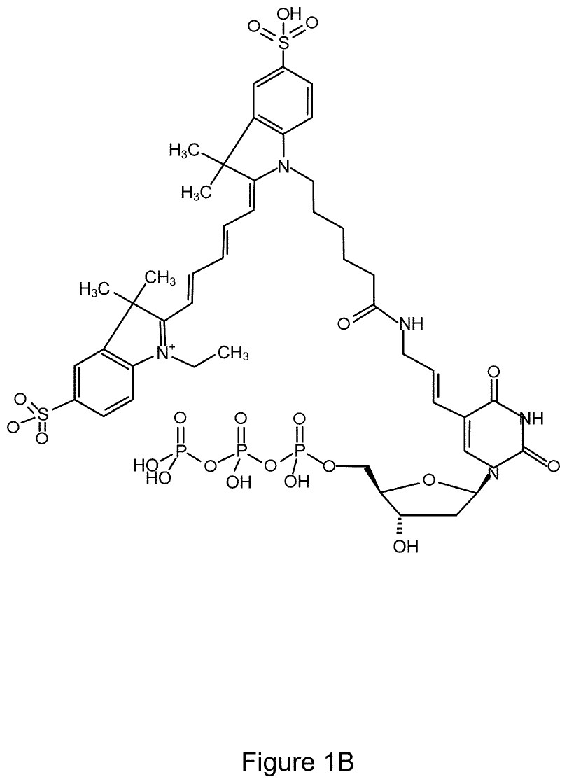

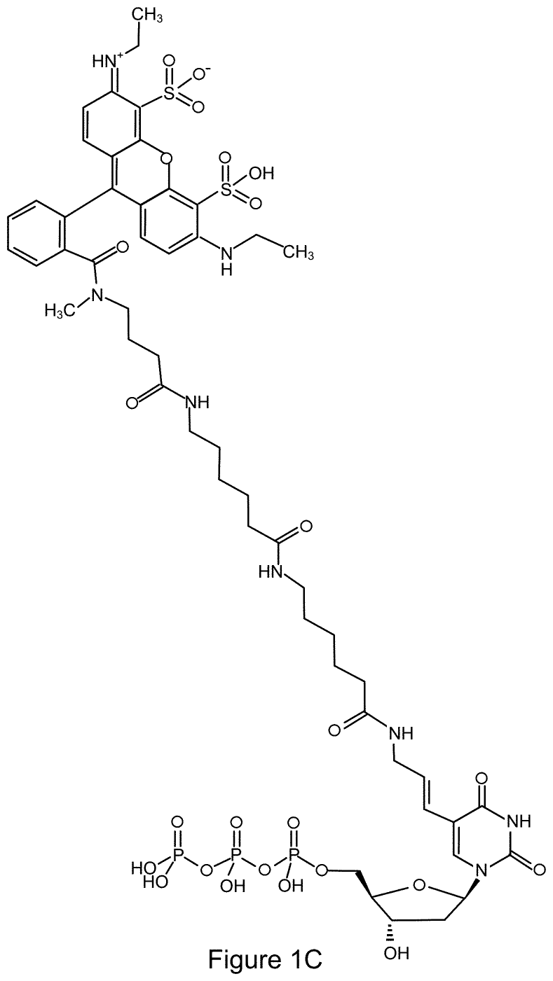

Direct label modified nucleotides (DLNs), are characterized by a linker that is covalently attached to the nucleobase on one end and a fluorescent dye or other chemical label on the opposite end (see FIG. 1A, FIG. 1B and FIG. 1C, which each depict embodiments of modified nucleotides, specifically, dUTP-label constructs which can be incorporated into DNA as substitutes for dTTP). The chemical label may be a charged or neutral molecule, hydrophobic or hydrophilic, or a combination of these characteristics. The linker moiety is typically comprised of a hydrocarbon chain of three or more carbon atoms. Often the chain will contain nitrogen and/or oxygen atoms. Many examples of modified nucleotides of this general form exist. Alternatively, the modification may be comprised of a linker-like molecule only. For example, the modification may be a polyethylene glycol (PEG) polymer. The key characteristic of DLNs is that the modification is complete and ready for detection once the nucleotide has been incorporated by polymerase during the amplification reaction.

Indirect Label Modified Nucleotides

Indirect label modified nucleotides (ILNs) also contain a carbon-based linker typically attached to the nucleobase. However, at the opposite end of the linker a chemically active group, like a primary amine, is present that can be used to attach other molecules to the linker post amplification. After the amplification reaction is complete, these modified nucleotides can be used to attach nanopore “detection” molecules directly to the newly synthesized DNA. Some examples of modified nucleotides of this form that are available include 1) bromo-2′-deoxyuridine-5′-triphosphate (BrdUTP), 2) 5-aminoallyl-2′-deoxyuridine-5′-triphosphate and 3) 5-ethynyl-2′-deoxyuridine-5′-triphosphate (EdUTP).

Bromouridine (BrdU) is detectable by a monoclonal antibody that is specific to bromouridine once it is incorporated into dsDNA. Binding of the antibody to the bromine--labeled DNA should result in a detectable change in the nanopore behavior as compared to the unbound antibody and unbound DNA.

Aminoallyl dUTP is detectable after incorporation into DNA via reaction of the free primary amino group with “detection” molecules labeled with N-hydroxysuccinimide using standard NHS coupling chemistry. N-hydroxysuccinimide-modified detection molecules, like high molecular weight PEG structures, fluorescent dyes or other charged or neutral molecules, can be used as labeling reagents. In this case, the label is attached to the DNA by a covalent bond, and hence can withstand harsher environments than non-covalent interactions like DNA hybridization probes or antibody-antigen interactions. Aminoallyl dUTP is readily incorporated by reverse transcriptase, Taq polymerase, phi29 DNA polymerase and Klenow fragment. This type of labeling has been used routinely for years to label cDNA prior to DNA microarray studies and has been shown to be very robust. In the case of microarray analysis, the NHS-labeling reagent is typically a fluorescent dye like Cy3 or Cy5.

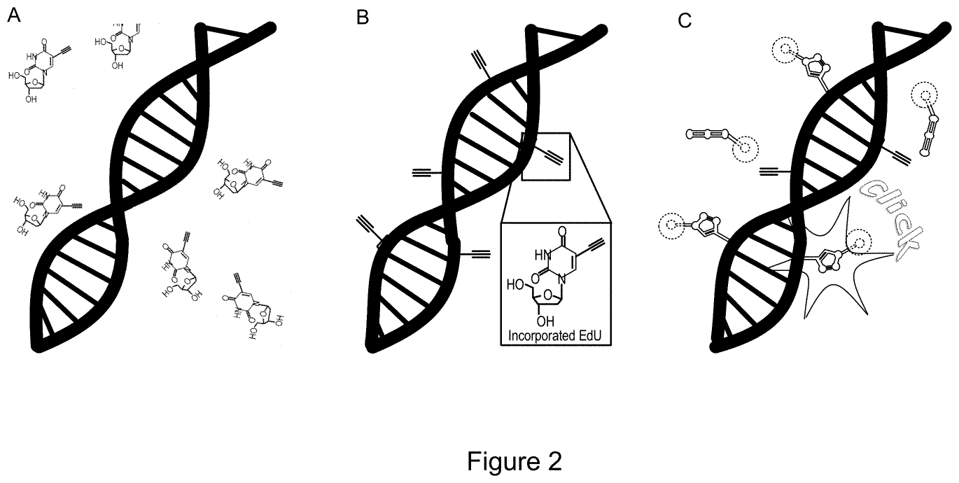

Ethynyl dUTP incorporation into DNA can be labeled using CLICK chemistry. Many versions of CLICK chemistry exist that are compatible with aqueous buffers like those used in the amplification reaction. Azide-modified detection molecules, like high molecular weight PEG structures for example, can be reacted with the newly synthesized DNA creating a labeled molecule that can be distinguished from other reaction components by the nanopore. See FIG. 2.

In some embodiments, a post amplification coupling reaction to couple a detection label to a modified nucleotide occurs on the nanopore strip in the same compartment that the amplification reaction occurs in. The anti-BrdU antibody described above in 1) could potentially be used in this manner. Alternatively, the coupling reagent may be positioned downstream of the amplification reaction where it becomes available for the coupling reaction as the DNA substrate is moved through the microfluidic channel towards the nanopore. Since the modified nucleotides will be incorporated at multiple sites in the DNA, the coupling reaction may not need to go to completion in order to get sufficient incorporation of the detection molecule. This can reduce the time needed for the coupling reaction to the time it takes for the DNA to travel from the amplification reaction compartment to the nanopore.

### Optimization and Multiplexing

In some embodiments, using either DLNs or ILNs, the number of labels on the DNA molecule we want to detect can be “dialed in” for optimal nanopore detectability. Incorporation of the modified nucleotides can occur at more than one position in the DNA (as illustrated in panel B or C of FIG. 2). The number of incorporated labels will depend on the relative concentration of the modified nucleotide to the concentration of the competing natural nucleotide, which will also be present in the amplification reaction. Hence, the main “dial” that controls the number of incorporations per DNA target is the ratio of modified nucleotide to natural nucleotide. This value can be optimized empirically. Incorporation of multiple labels on the DNA can increase the interaction of these molecules with the nanopore and thus the resolution of detection of target polynucleotides. Furthermore, different types of chemical labels can have distinctly different behavior in the nanopore. By utilizing combinations, or cocktails, of differently labeled nucleotides in the amplification reaction, the behavior of labeled nucleic acid in the nanopore can be further enhanced compared to a single type of label. These labeling cocktails may include different labels attached to the same nucleobase, the same label attached to two or more different nucleobases, or a combination of both.

Another advantage of this approach is the broad compatibility with any nucleic acid amplification reaction. One set of labeling reagents can be used for several different types of nucleic acid assays, whether the target is RNA or DNA. The same primer sequences and amplification conditions that have already been optimized and validated can be used in this approach with minimal or no modification.

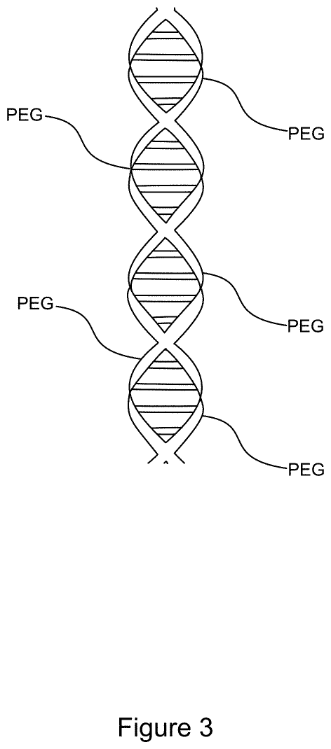

In some embodiments, detection molecules include highly branched and large molecular weight PEG structures. In some embodiments, large and complex PEG structures may not be required because the labeling of the DNA target can occur at more than one site. FIG. 3 illustrates what DNA labeled as described in this proposal may look like, according to some embodiments. In FIG. 3, linear PEG molecules extend perpendicular to the axis of the double stranded DNA creating “bristle”-like structures that can interact favorably with the nanopore as the DNA is translocated through the pore. The “bristles” may be PEG structures as shown, or any other type of label that sufficiently alters a detectable signal generated by the polynucleotide comprising modified nucleotides in the nanopore.

### Nanopore Devices

A nanopore device, as provided, includes at least a pore that forms an opening in a structure separating an interior space of the device into two volumes, and at least a sensor configured to identify objects (for example, by detecting changes in parameters indicative of objects) passing through the pore. Nanopore devices used for the methods described herein are also disclosed in PCT Publication WO/2013/012881, incorporated by reference in entirety.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the nanopore device further includes means to move a polynucleotide across the pore and/or means to identify objects that pass through the pore.

In one embodiment, the nanopore device includes a plurality of chambers, each chamber in communication with an adjacent chamber through at least one pore. Further, the device includes a sensor at each pore capable of identifying the target polynucleotide during the movement. In one aspect, the identification entails identifying individual components of the target polynucleotide. In another aspect, the identification entails identifying modified nucleotides incorporated in the target polynucleotide. When a single sensor is employed, the single sensor may include two electrodes placed at both ends of a pore to measure an ionic current across the pore. In another embodiment, the single sensor comprises a component other than electrodes.

In some aspects, the device further includes means to move a target polynucleotide from one chamber to another.

In accordance with one embodiment of the present disclosure, provided is a device comprising an upper chamber, a middle chamber and a lower chamber, wherein the upper chamber is in communication with the middle chamber through a first pore, and the middle chamber is in communication with the lower chamber through a second pore. Such a device may have any of the dimensions or other characteristics previously disclosed in U.S. Publ. No. 2013-0233709, entitled Dual-Pore Device, which is herein incorporated by reference in its entirety.

In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, each pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the pore has a substantially round shape. “Substantially round”, as used here, refers to a shape that is at least about 80 or 90% in the form of a cylinder. In some embodiments, the pore is square, rectangular, triangular, oval, or hexangular in shape.

In one aspect, the pore has a depth that is between about 1 nm and about 10,000 nm, or alternatively, between about 2 nm and about 9,000 nm, or between about 3 nm and about 8,000 nm, etc.

In some aspects, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials. Nanopores are sized to permit passage through the pore of the polynucleotide comprising modified nucleotides. In other embodiments, temporary blockage of the pore may be desirable for discrimination of molecule types.

In some aspects, the length or depth of the nanopore is sufficiently large so as to form a channel connecting two otherwise separate volumes. In some such aspects, the depth of each pore is greater than 100 nm, 200 nm, 300 nm, 400 nm, 500 nm, 600 nm, 700 nm, 800 nm, or 900 nm. In some aspects, the depth of each pore is no more than 2000 nm or 1000 nm.

In one aspect, the device has electrodes in the chambers connected to one or more power supplies. In some aspects, the power supply includes a voltage-clamp or a patch-clamp, which can supply a voltage across each pore and measure the current through each pore independently.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO₂, HfO₂, Al₂O₃, or other metallic layers, or any combination of these materials. In some aspects, for example, a single sheet of graphene membrane of about 0.3 nm thick can be used as the pore-bearing membrane.

Devices that are microfluidic and that house nanopore microfluidic chip implementations can be made by a variety of means and methods. For a microfluidic chip comprised nanopore membranes, membranes can be drilled by a single beam to form nanopores, though using different beams on each side of the membranes is also possible in concert with any suitable alignment technique. In general terms, the housing ensures sealed separation of chambers on either side of each nanopore.

More specifically, the pore-bearing membranes can be made with transmission electron microscopy (TEM) grids with a 5-100 nm thick silicon, silicon nitride, or silicon dioxide windows. Spacers can be used to separate the membranes, using an insulator, such as SU-8, photoresist, PECVD oxide, ALD oxide, ALD alumina, or an evaporated metal material, such as Ag, Au, or Pt, and occupying a small volume within the otherwise aqueous portion of Chamber B between the membranes.

A focused electron or ion beam can be used to drill pores through the membranes, naturally aligning them. The pores can also be sculpted (shrunk) to smaller sizes by applying a correct beam focusing to each layer. Any single nanopore drilling method can also be used to drill the pair of pores in the two membranes, with consideration to the drill depth possible for a given method and the thickness of the membranes. Predrilling a micro-pore to a prescribed depth and then a nanopore through the remainder of the membranes is also possible to further refine the membrane thickness.

By virtue of the voltages present at the pores of the device, charged molecules can be moved through each pore between chambers. Speed and direction of the movement can be controlled by the magnitude and polarity of the voltages.

As discussed above, in various aspects, the nanopore device further includes one or more sensors to carry out the detection of the target polynucleotide.

The sensors used in the device can be any sensor suitable for identifying a target polynucleotide amplicon comprising one or more modified nucleotides. For instance, a sensor can be configured to identify the target polynucleotide by measuring a current, a voltage, a pH value, an optical feature, or residence time associated with the polymer. In other aspects, the sensor may be configured to identify one or more individual components of the target polynucleotide or one or more components bound or attached to the target polynucleotide. The sensor may be formed of any component configured to detect a change in a measurable parameter where the change is indicative of the target polynucleotide, a component of the target polynucleotide, or preferably, a component bound or attached to the target polynucleotide. In one aspect, the sensor includes a pair of electrodes placed at two sides of a pore to measure an ionic current across the pore when a molecule or other entity, in particular a target polynucleotide, moves through the pore. In certain aspects, the ionic current across the pore changes measurably when a target polynucleotide segment passing through the pore is bound to a payload molecule. Such changes in current may vary in predictable, measurable ways corresponding with, for example, the presence, absence, and/or size of the target polynucleotide molecule present.

In a preferred embodiment, the sensor comprises electrodes that apply voltage and are used to measure current across the nanopore. Translocations of molecules through the nanopore provides electrical impedance (Z) which affects current through the nanopore according to Ohm's Law, V=IZ, where V is voltage applied, I is current through the nanopore, and Z is impedance. Inversely, the conductance G=1/Z are monitored to signal and quantitate nanopore events. The result when a molecule translocates through a nanopore in an electrical field (e.g., under an applied voltage) is a current signature that may be correlated to the molecule passing through the nanopore upon further analysis of the current signal.

When residence time measurements from the current signature are used, the size of the component can be correlated to the specific component based on the length of time it takes to pass through the sensing device.

In one embodiment, a sensor is provided in the nanopore device that measures an optical feature of the polymer, a component (or unit) of the polymer, or a component bound or attached to the polymer. One example of such measurement includes the identification of an absorption band unique to a particular unit by infrared (or ultraviolet) spectroscopy.

In some embodiments, the sensor is an electric sensor. In some embodiments, the sensor detects a fluorescent signature. A radiation source at the outlet of the pore can be used to detect that signature.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1: Nanopore Detection and Discrimination of DNA and DNA-DUTP-Atto-488

279bp or 466bp DNA fragments with or without dUTP-modified nucleotides (Jena Bioscience) were amplified with Taq polymerase using the following PCR cycling protocol:

Step 1: 94° C.—2 min

Step 2: 94° C.—30 s

Step 3: 60° C.—30 s

Step 4: 72° C.—1 min
Steps 2-4 repeated 34×

Step 5: 72° C. 5 min

Step 6: 4° C. hold

Following amplification, the DNA products were purified using a DNA cleanup kit (Viogene®). The concentrations of the purified DNA amplicons were measured with a NanoDrop™ spectrophotometer (Thermo Fisher Scientific™) and subsequently verified via an electrophoretic mobility shift assay (EMSA). The EMSA assay was completed in a 6% polyacrylamide gel (Invitrogen™) for 30 minutes at 150V. The gel was stained with SybrGreen and visualized using a Gel Doc EZ Imager (Bio-Rad®).

Nanopore chips containing nanopores ranging from 30-45 nm in a SiN membrane were fabricated and characterized at the Stanford Nanofabrication Facility. Experiments were conducted at room temperature in a 2M lithium chloride solution containing a final concentration of 10 mM Tris buffer and 1 mM EDTA (pH 8.8). Experimental samples suspended in nanopore running buffer were introduced to the voltage negative chamber (cis side) and a voltage of 100 mV was applied to translocate the molecules through the nanopore to the trans side fluidic chamber. The translocation event signals were characterized and plotted according to their dwell time in the nanopore (duration, seconds), in addition to the change in conductance during the translocation (delta G, nanosiemens).

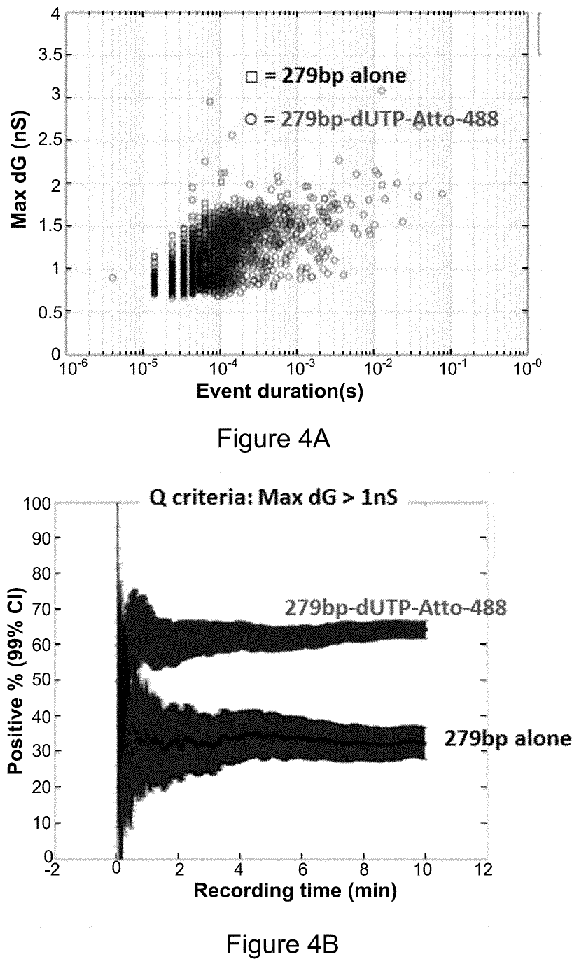

A 279 bp DNA fragment alone was analyzed with a 32 nm nanopore (5 nM, 100 mV, 2M LiCl, 10 mM Tris, 1 mM EDTA, pH 8.8), producing 744 events in 10 minutes (FIG. 4A). Few events hit a depth of at least 1 nS (18.0%, FIG. 4B). Following removal of the 279bp DNA from the chamber adjacent to the nanopore, 5 nM of 279bp DNA-dUTP-Atto-488 was added, where DNA-dUTP-Atto488 here refers to a 279bp DNA strand with dUTP-Atto-488 (FIG. 1A) incorporated into the DNA backbone. By adding the dUTP-Atto-488 to the DNA strand, the net size of the DNA strand increased, thereby increasing the max dG of the population in comparison to 279bp alone. The bulkier 279bp DNA-dUTP-Atto-488 reagent produced 2582 events over 10 minutes, with an increase to 64.5% of events hitting a depth of at least 1 nS (FIG. 4B). Thus, nanopore detection as described herein can be used to detect the presence of the target polynucleotide including the dUTP-Atto-488 modified nucleotide with 99% confidence

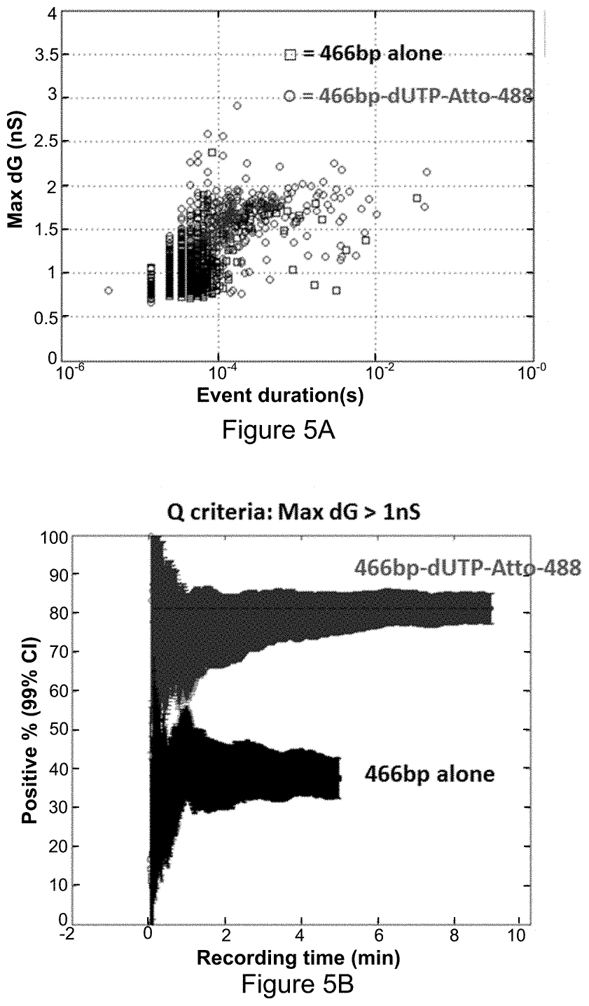

dUTP-Atto-488 was also incorporated into a 466bp DNA fragment and analyzed analogous to the 279bp fragments (FIG. 5A). 1 nM of 466bp DNA alone was measured with a 29 nm nanopore, resulting in 585 events over the course of 5 minutes. Of those 585 events, a modest 37.4% of them produced a max dG of 1 nS or greater. However, following incorporation of dUTP-Atto-488 into the 466bp DNA fragment, 81.2% of the 681 events collected over the course of 9 minutes reached the same 1 nS threshold (FIG. 5B). Thus, nanopore detection as described herein can be used to detect the presence of the target polynucleotide including the dUTP-Atto-488 modified nucleotide with 99% confidence.

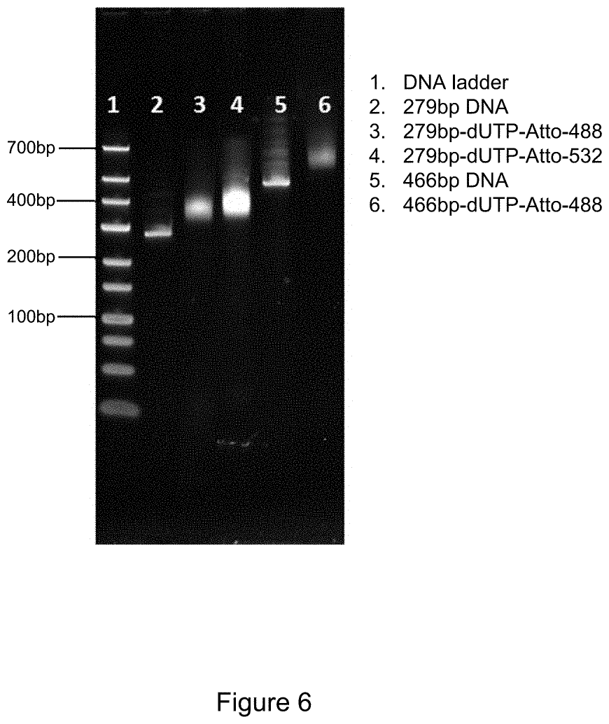

FIG. 6 shows the results of an electrophoretic mobility shift assay (EMSA) of the amplification products used in nanopore experimentation. FIG. 6 depicts 279bp DNA without modification (lane 2) in addition to 279bp with dUTP-Atto-488 incorporated (lane 3). A shift up in the gel matrix indicates successful incorporation of dUTP modifications. Successful incorporation of dUTP-Atto-488 in a 466bp amplicon is also shown (lane 6).

### Example 2: Nanopore Detection and Discrimination of DNA and DNA-DUTP-Cy5 or DNA-dUTP-532

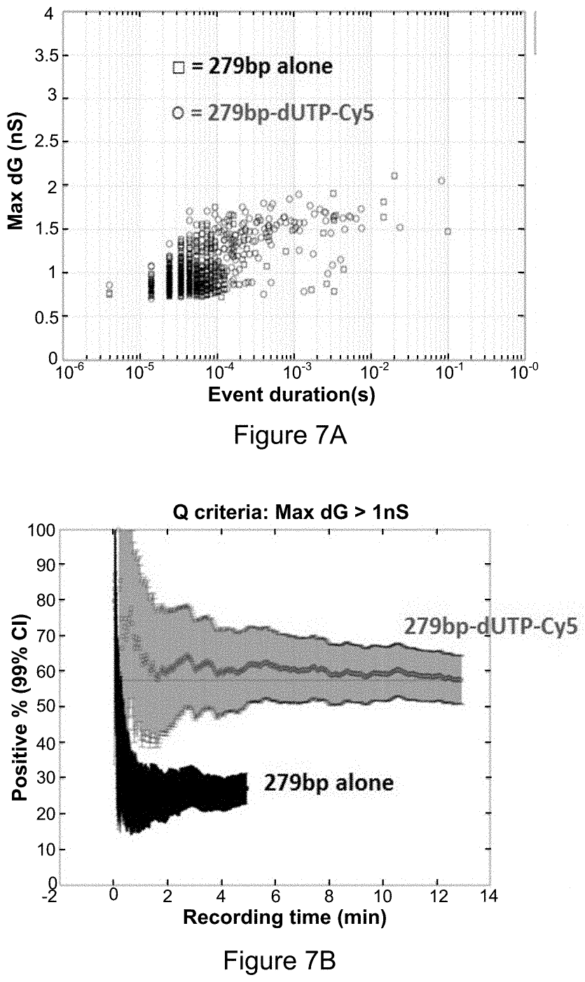

A 279bp DNA fragment was analyzed in a 32 nm nanopore and compared to 279bp DNA with dUTP-Cy5 or dUTP-Atto-532 incorporated into the DNA strand. The 279bp of DNA alone produced 744 events over the course of 10 minutes (FIG. 7A) with 18.0% of those events reaching a max dG of 1 nS or greater. Following removal of the 279bp DNA from the chamber adjacent to the nanopore, 5 nM of 279bp DNA-dUTP-Cy5 was added. The 279bp strand with the dUTP-Cy5 incorporated produced 515 events over 7 minutes with 49.1% of those events reaching a max dG of 1 nS or greater (FIG. 7B). Thus, nanopore detection as described herein can be used to detect the presence of the target polynucleotide including the dUTP-Cy5 modified nucleotide with 99% confidence.

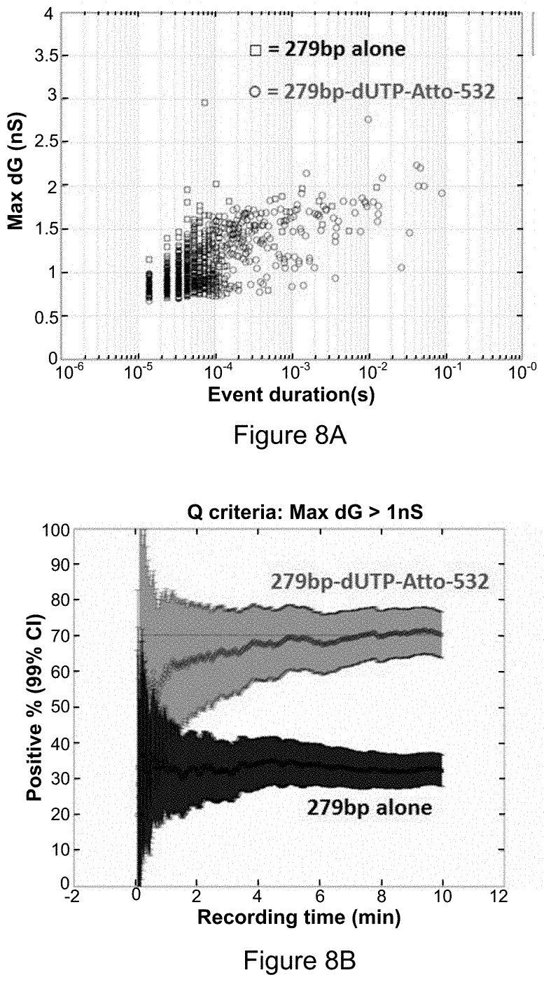

Separately, dUTP-Atto-532 was synthesized into the backbone of the 279bp amplicon. Following removal of the previous sample, 5 nM of the DNA-dUTP-Atto-532 was added. Similar to other tags, the net increase in size of the DNA strand by modified dUTP incorporation rendered a population with a greater percentage of events reaching a max dG of 1 nS or greater thereby indicating the presence of the target polynucleotide with 99% confidence. Of the 1375 events collected over 10 minutes of analysis (FIG. 8A), 59.3% of those events reached the 1 nS threshold compared to the 18.0% without the modified dUTP incorporation (FIG. 8B). Thus, nanopore detection as described herein can be used to detect the presence of the target polynucleotide including the dUTP-Atto-532 modified nucleotide with 99% confidence.

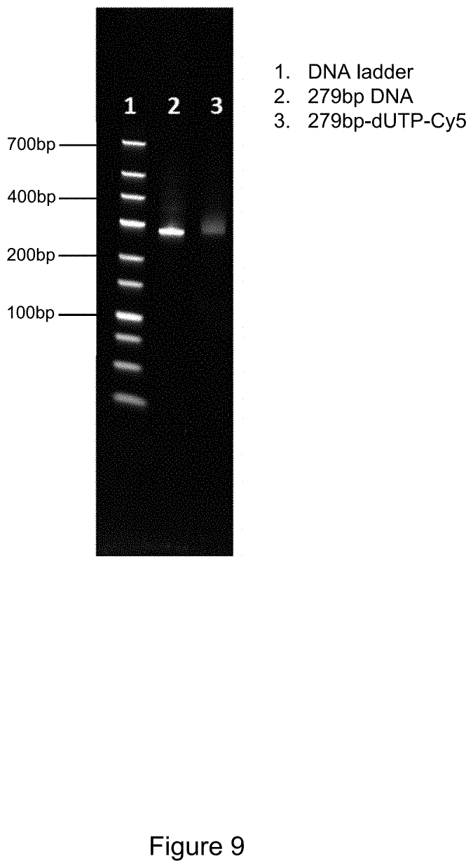

FIG. 6 and FIG. 9 show the results of an electrophoretic mobility shift assay (EMSA) of the amplification products used in nanopore experimentation. FIG. 6A depicts 279bp DNA without modification (lane 2) in addition to 279bp with dUTP-Atto-532 incorporated (lane 4). A shift up in the gel matrix indicates successful incorporation of dUTP modifications. FIG. 9 shows 279bp DNA without modification in addition to the same fragment with dUTP-Cy5 incorporated (lanes 2 and 3, respectively).

1. A method of determining the presence or absence of a target polynucleotide suspected of being present in a sample, comprising:
providing a sample suspected of comprising a target polynucleotide;
providing one or more modified nucleotides;
performing a template-driven polymerization reaction on said sample to incorporate said one or more modified nucleotides into a growing strand complementary to said target polynucleotide, if present, to generate an polynucleotide reaction product comprising said one or more modified nucleotides;
loading said sample into a device comprising a nanopore, wherein said nanopore separates an interior space of the device into two volumes;
configuring the device to pass the polynucleotide reaction product, if present, through said nanopore, wherein the device comprises a sensor configured to detect an electrical signal generated by objects passing through the nanopore; and
detecting the presence or absence of said target polynucleotide in said sample by determining whether a polynucleotide comprising said one or more modified nucleotides passed through the nanopore using said electrical signal from the sensor.

2. The method of claim 1, wherein said one or more modified nucleotides comprises a direct label modified nucleotide or an indirect label modified nucleotide.

3. The method of claim 1, wherein said one or more modified nucleotides comprises a charged chemical moiety, a neutral chemical moiety, a hydrophobic moiety, or a hydrophilic moiety.

4. The method of claim 1, wherein said one or more modified nucleotides comprises a linker capable of binding to a charged chemical moiety, a neutral chemical moiety, a hydrophobic moiety, or a hydrophilic moiety.

5. The method of claim 1, wherein said one or more modified nucleotides comprises a fluorescent dye.

6. The method of claim 1, wherein said one or more modified nucleotides comprises Atto488, Atto425, Atto532, Cy5, Texas Red, Fluorescein-12, Rhodamine-12, or aminomethylcoumarin-6.

7. The method of claim 1, wherein said modified nucleotide is a dUTP, dTTP, dCTP, dATP, or dGTP.

8. The method of claim 1, wherein said one or more modified nucleotides comprises a polyethylene glycol.

9. The method of claim 1, wherein said one or more modified nucleotides comprise a linker.

10. The method of claim 9, wherein said linker comprises bromo-2′-deoxyuridine-5′-triphosphate (BrdUTP), 5-aminoallyl-2′-deoxyuridine-5′-triphosphate, or 5-ethynyl-2′-deoxyuridine-5′-triphosphate (EdUTP), N6-(6-amino)hexyl-dATP (or dUTP or dCTP) and 7-propargylamino-7-deaza-dATP (or dUTP or dCTP).

11. The method of any one of claim 9 or 10, further comprising binding a detectable moiety to said linker, wherein said electrical signal is modified by the presence of said detectable moiety.

12. The method of claim 11, wherein said detectable moiety comprises an antibody.

13. The method of claim 11, wherein said detectable moiety comprises N-hydroxysuccinimide.

14. The method of claim 11, wherein said detectable moiety is azide-modified.

15. The method of claim 1, wherein the sensor measures an electrical signal that fluctuates upon translocation of said polynucleotide reaction product through said nanopore.

16. The method of claim 1, comprising detecting a plurality of target polynucleotides suspected of being present in the same sample by generating a plurality of distinct polynucleotide reaction products.

17. The method of claim 16, wherein said electrical signal is distinct for each of said plurality of target polynucleotides.

18. The method of claim 17, wherein said plurality of distinct polynucleotide reaction products are of different lengths.

19. The method of claim 17, wherein said plurality of distinct polynucleotide reaction products each comprise a unique modified nucleotide.

20. The method of claim 17, wherein said plurality of distinct polynucleotide reaction products comprise a unique modified nucleotide incorporation pattern.

21. The method of claim 16, wherein said template-driven polymerization reaction comprises a plurality of sequence-specific primers to enable multiplexed amplification and nanopore detection of more than one target.

22. The method of claim 1, wherein said polynucleotide is DNA or RNA.

23. The method of claim 1, wherein said template-driven polymerization reaction is an amplification reaction.

24. The method of claim 23, wherein said amplification reaction is a polymerase chain reaction or an isothermal reaction.

25. The method of claim 1, wherein said template-driven polymerization reaction comprises a polymerase.

26. The method of claim 25, wherein said polymerase is DNA polymerase or a reverse transcriptase.

27. The method of claim 25, wherein said polymerase is a thermostable DNA polymerase or a thermolabile DNA polymerase.

28. The method of claim 1, wherein the modified nucleotide comprises a plurality of labels.

29. The method of claim 1, wherein said polynucleotide reaction product is from 200 to 500 bases, from 100 to 2,000 bases, or from 50 to 10,000 bases in length.

30. The method of claim 1, wherein said polynucleotide reaction product is greater than 50 bases, greater than 100 bases, greater than 200 bases, greater than 300 bases, or greater than 400 bases in length.

31. The method of claim 1, wherein said polynucleotide reaction product is less than 50,000 bases, less than 10,000 bases, less than 5,000 bases, less than 1,000 bases, or less than 500 bases in length.

32. A method of identifying a modified nucleotide present in a polynucleotide, comprising:
loading a sample suspected of comprising a polynucleotide comprising a plurality of modified nucleotides into a device comprising a nanopore, wherein said nanopore separates an interior space of the device into two volumes;
configuring the device to pass the polynucleotide, if present, through said nanopore, wherein the device comprises a sensor configured to detect an electrical signal generated by objects passing through the nanopore, and wherein said polynucleotide comprising said modified nucleotides generates a distinct electrical signal from a polynucleotide without said modified nucleotides; and
detecting the presence or absence of said polynucleotide in said sample by determining whether said polynucleotide comprising said plurality of modified nucleotides passed through the nanopore using said electrical signal detected by the sensor.

33. The method of claim 32, wherein said polynucleotide comprises at least 5, at least 10, at least 20, at least 30, at least 40, at least 50, at least 100, at least 200, or at least 500 modified nucleotides.

34. The method of claim 32, wherein said plurality of modified nucleotides comprise a direct label modified nucleotide or an indirect label modified nucleotide.

35. The method of claim 32, wherein said plurality of modified nucleotides comprise a charged chemical moiety, a neutral chemical moiety, a hydrophobic moiety, or a hydrophilic moiety.

36. The method of claim 32, wherein said plurality of modified nucleotides comprise a linker capable of binding to a charged chemical moiety, a neutral chemical moiety, a hydrophobic moiety, or a hydrophilic moiety.

37. The method of claim 32, wherein said plurality of modified nucleotides comprise a fluorescent dye.

38. The method of claim 32, wherein said plurality of modified nucleotides comprise Atto488, Atto425, Atto532, Cy5, Texas Red, Fluorescein-12, Rhodamine-12, or aminomethylcoumarin-6.

39. The method of claim 32, wherein said plurality of modified nucleotides comprise dUTP, dTTP, dCTP, dATP, or dGTP.

40. The method of claim 32, wherein said plurality of modified nucleotides comprise a polyethylene glycol.

41. The method of claim 32, wherein said plurality of modified nucleotides comprise a linker.

42. The method of claim 41, wherein said linker comprises bromo-2′-deoxyuridine-5′-triphosphate (BrdUTP), 5-aminoallyl-2′-deoxyuridine-5′-triphosphate, or 5-ethynyl-2′-deoxyuridine-5′-triphosphate (EdUTP), N6-(6-amino)hexyl-dATP (or dUTP or dCTP) and 7-propargylamino-7-deaza-dATP (or dUTP or dCTP).

43. The method of any one of claim 41 or 42, further comprising binding a detectable moiety to said linker, wherein said electrical signal is modified by the presence of said detectable moiety.

44. The method of claim 43, wherein said detectable moiety comprises an antibody.

45. The method of claim 43, wherein said detectable moiety comprises N-hydroxysuccinimide.

46. The method of claim 43, wherein said detectable moiety is azide-modified.

47. The method of claim 32, wherein the sensor measures an electrical signal that fluctuates upon translocation of said polynucleotide reaction product through said nanopore.

48. The method of claim 32, comprising detecting a plurality of distinct polynucleotides comprising modified nucleotides.

49. The method of claim 48, wherein said electrical signal is distinct for each of said plurality of polynucleotides.

50. The method of claim 48, wherein said plurality of distinct polynucleotides are of different lengths.

51. The method of claim 48, wherein said plurality of distinct polynucleotides each comprise a unique modified nucleotide.

52. The method of claim 48, wherein said plurality of distinct polynucleotides have a distinct pattern of modified nucleotides along the length of each distinct polynucleotide.

53. The method of claim 32, wherein said polynucleotide is DNA or RNA.

54. The method of claim 32, wherein the modified nucleotide comprises a plurality of labels.

55. The method of claim 32, wherein said polynucleotide is from 200 to 500 bases, from 100 to 2,000 bases, or from 50 to 10,000 bases in length.

56. The method of claim 32, wherein said polynucleotide is greater than 50 bases, greater than 100 bases, greater than 200 bases, greater than 300 bases, or greater than 400 bases in length.

57. The method of claim 32, wherein said polynucleotide is less than 50,000 bases, less than 10,000 bases, less than 5,000 bases, less than 1,000 bases, or less than 500 bases in length.

58. The method of claim 32, wherein said modified nucleotides are individually incorporated into said polynucleotide by an amplification reaction.

59. A system, comprising:
a device comprising a nanopore, wherein said nanopore separates an interior space of the device into two volumes, wherein the device comprises a sensor configured to detect an electrical signal generated by objects passing through the nanopore;
a polynucleotide comprising a modified nucleotide, wherein said polynucleotide is loaded into said device for detection by voltage-induced translocation through said nanopore; and
a module for analyzing said electrical signal to detect the presence or absence of said polynucleotide comprising said modified nucleotide.

60. The system of claim 59, wherein said polynucleotide comprises a plurality of modified nucleotides.

61. The system of claim 59, wherein said polynucleotide comprises at least 5, at least 10, at least 20, at least 30, at least 40, at least 50, at least 100, at least 200, or at least 500 modified nucleotides.

62. The system of claim 59, wherein said modified nucleotide comprises a direct label modified nucleotide or an indirect label modified nucleotide.

63. The system of claim 59, wherein said modified nucleotide comprises a charged chemical moiety, a neutral chemical moiety, a hydrophobic moiety, or a hydrophilic moiety.

64. The system of claim 59, wherein said modified nucleotide comprises a linker capable of binding to a charged chemical moiety, a neutral chemical moiety, a hydrophobic moiety, or a hydrophilic moiety.

65. The system of claim 59, wherein said modified nucleotide comprises a fluorescent dye.

66. The system of claim 59, wherein said modified nucleotide comprises Atto488, Atto425, Atto532, Cy5, Texas Red, Fluorescein-12, Rhodamine-12, or aminomethylcoumarin-6.

67. The system of claim 59, wherein said plurality of modified nucleotide is dUTP, dTTP, dCTP, dATP, or dGTP.

68. The system of claim 59, wherein said modified nucleotide comprises a polyethylene glycol.

69. The system of claim 59, wherein said modified nucleotide comprise a linker.

70. The system of claim 69, wherein said linker comprises bromo-2′-deoxyuridine-5′-triphosphate (BrdUTP), 5-aminoallyl-2′-deoxyuridine-5′-triphosphate, or 5-ethynyl-2′-deoxyuridine-5′-triphosphate (EdUTP), N6-(6-amino)hexyl-dATP (or dUTP or dCTP) and 7-propargylamino-7-deaza-dATP (or dUTP or dCTP).

71. The system of claim 69 or 70, said linker is bound to a detectable moiety, and wherein said electrical signal is modified by the presence of said detectable moiety.

72. The system of claim 71, wherein said detectable moiety comprises an antibody.

73. The system of claim 71, wherein said detectable moiety comprises N-hydroxysuccinimide.

74. The system of claim 71, wherein said detectable moiety is azide-modified.

75. The system of claim 59, wherein the sensor is configured to measure an electrical signal that fluctuates upon translocation of said polynucleotide reaction product through said nanopore.

76. The system of claim 59, wherein said polynucleotide is DNA or RNA.

77. The system of claim 59, wherein the modified nucleotide comprises a plurality of labels.

78. The system of claim 59, wherein said polynucleotide is from 200 to 500 bases, from 100 to 2,000 bases, or from 50 to 10,000 bases in length.

79. The system of claim 59, wherein said polynucleotide is greater than 50 bases, greater than 100 bases, greater than 200 bases, greater than 300 bases, or greater than 400 bases in length.

80. The system of claim 59, wherein said polynucleotide is less than 50,000 bases, less than 10,000 bases, less than 5,000 bases, less than 1,000 bases, or less than 500 bases in length.
